(19) United States
(12) Patent Application Publication
(10) Pub. No.: US 2023/0292696 A1
(43) Pub. Date: Sep. 21, 2023

(54) Therapeutic Animal Crate

(71) Applicants: Scott Nelson, Mission Viejo, CA (US); Troy Stites, Dallas, TX (US)

(72) Inventors: Scott Nelson, Mission Viejo, CA (US); Troy Stites, Dallas, TX (US)

(21) Appl. No.: 17/992,901

(22) Filed: Nov. 22, 2022

Related U.S. Application Data
(63) Continuation of application No. 17/700,465, filed on Mar. 21, 2022, now abandoned.

(51) Int. Cl. A01K 1/00; A61N 5/06; A61N 2/00; A01K 1/035

(52) U.S. Cl. CPC A01K 1/0035; A01K 1/035; A61N 2/002; A61N 5/06; A61N 2005/0627; A61N 2005/0632; A61N 2005/0661; A61N 2005/0663

(57) ABSTRACT

This disclosure relates to a therapeutic crate system for housing an animal, the crate system comprising a crate housing including a top wall, a bottom wall located opposite the top wall, and side walls extending between the top wall and the bottom wall. In some embodiments, the therapeutic crate system comprises a sensor coupled to the crate housing, the sensor configured to determine a distance between the animal and the sensor. According to some embodiments, the therapeutic crate system comprises an energized panel coupled to a wall selected from the group consisting of the top wall, the bottom wall, and at least one of the side walls, and operatively coupled to the sensor, the energized panel configured to deliver a type of therapy selected from the group consisting of phototherapy, PEMF treatments, and sound therapy.

### BACKGROUND

### Field of the Invention

Various embodiments disclosed herein describe an animal crate. More specifically, various embodiments disclosed herein describe an animal crate that provides therapy.

### Description of Related Art

Therapeutic treatments for humans are plentiful and diverse. However, these same therapeutic treatments are generally unavailable for animals. Systems and methods in the prior art have attempted to bring these various treatments to animals, but lack a convenient method of containing the animal to efficiently provide a method or methods of therapy. Thus, there is a need for systems and methods to remedy these deficiencies found in the prior art.

### BRIEF SUMMARY OF THE INVENTION

This disclosure relates to a therapeutic crate system for housing an animal, the crate system comprising a crate housing including a top wall, a bottom wall located opposite the top wall, and side walls extending between the top wall and the bottom wall. In some embodiments, the therapeutic crate system comprises a sensor coupled to the crate housing, the sensor configured to determine a distance between the animal and the sensor. According to some embodiments, the therapeutic crate system comprises an energized panel coupled to a wall selected from the group consisting of the top wall, the bottom wall, and at least one of the side walls, and operatively coupled to the sensor, the energized panel configured to deliver a type of therapy selected from the group consisting of phototherapy, PEMF treatments, and sound therapy.

The energized panel may be configured to adjust light intensity based on the distance of the animal from the sensor. In some embodiments, the energized panel is configured to detach from the wall. Additional embodiments will further comprising an energized panel or mat coupled to the bottom wall.

According to some embodiments, the energized panel comprises a first energized panel, the therapeutic crate system further comprising a second energized panel located along a wall selected from the group consisting of the top wall, the bottom wall, and at least one of the side walls, wherein the first energized panel and the second energized panel are not located on a same wall. The second energized panel may deliver a therapy selected from the group consisting of PEMF therapy and sound therapy.

In some embodiments, the crate housing comprises a portable housing. According to some embodiments, the crate housing comprises a fixed housing. Portable pet crates can be folded from a three-dimensional, rectangular box to a flat configuration for storing and moving while occupying a fractional volume of space in contrast to the in-use configuration needed to house an animal while being self-supporting. Generally, portable exemplary embodiments shall have a structural, solid frame and fabric or hinged sides to change from transit, low volume form to static, animal holding form in a short amount of time without the use of tools.

The therapeutic crate system may further comprise a controller operatively coupled to the energized panel, the controller configured to establish a programable element selected from the group consisting of treatment time, treatment modality, light flux, PEMF therapy settings, and sound therapy settings. In some embodiments, the treatment time is between one second and twenty-four hours. According to some embodiments, the light flux is between 0.01 lux and 100,000 lux.

This disclosure also describes a software-enabled therapeutic crate system comprising a crate housing including a top wall, a bottom wall located opposite the top wall, and side walls extending between the top wall and the bottom wall. In some embodiments, the software-enabled therapeutic crate system comprises a sensor coupled to the crate housing, the sensor configured to determine a distance between an animal and the sensor. According to some embodiments, the software-enabled therapeutic crate system comprises an energized panel coupled to a wall selected from the group consisting of the top wall, the bottom wall, and at least one of the side walls, the energized panel operatively coupled to the sensor. The software-enabled therapeutic crate system may comprise a software application configured to run on a remote computing device, wherein the software application is coupled communicatively to the energized panel.

In some embodiments, the software application is configured to perform an action selected from the group consisting of changing an amount of time on a timer and remotely unlocking a locking mechanism. According to some embodiments, the energized panel is configured to deliver a type of therapy selected from the group consisting of phototherapy, PEMF treatments, and sound therapy. The software application may be configured to change a programmable component selected from the group consisting of optical intensity delivered by the energized panel, light wavelengths delivered by the energized panel, and the type of therapy delivered by the energized panel.

In some embodiments, the software-enabled therapeutic crate system further comprises a camera coupled to a wall selected from the group consisting of the top wall, the bottom wall, and at least one of the side walls, the camera coupled communicatively to the software application, wherein the camera is configured to communicate information related to a component selected from the group consisting of a type of animal, a breed of animal, a weight of the animal, a height of the animal, a length of the animal, a hair color of the animal, and a hair length of the animal. According to some embodiments, the software application is programmable with respect to the component selected from the group consisting of the type of animal, the breed of animal, the weight of the animal, the height of the animal, the length of the animal, the hair color of the animal, and the hair length of the animal. The software application may be configured to establish a programable element selected from the group consisting of treatment time, treatment modality, light flux, PEMF therapy settings, and sound therapy settings.

In some embodiments, the software-enabled therapeutic crate system further comprises a speaker coupled to the crate housing, the speaker coupled communicatively to the software application, wherein the software application is configured to emit a sound selected from the group consisting of communication to an animal located inside the crate housing and sound therapy from the speaker. According to some embodiments, the software application is coupled communicatively to the sensor. The sensor may be configured to provide information about a distance from the energized panel to the animal to the software application. In some embodiments, the software application is configured to adjust a light intensity of the energized panel.

### BRIEF DESCRIPTION OF THE DRAWINGS

Features, aspects, and advantages are described below with reference to the drawings, which are intended to illustrate, but not to limit, the invention. In the drawings, like reference characters denote corresponding features consistently throughout similar embodiments.

FIG. 1 illustrates a profile view of a therapeutic animal crate with closed doors, according to some embodiments.

FIG. 2 illustrates an exploded profile view of the therapeutic animal crate, according to some embodiments.

FIG. 3 illustrates a profile view of the therapeutic animal crate with open doors, according to some embodiments.

FIG. 4 illustrates a front view of the therapeutic animal crate with closed doors, according to some embodiments.

FIG. 5 illustrates a front view of an energized panel, according to some embodiments.

FIGS. 6A and 6B illustrate profile views of the therapeutic animal crate with open doors, according to some embodiments.

FIGS. 7A and 7B illustrate front views of the therapeutic animal crate with open doors, according to some embodiments.

FIG. 8 illustrates a profile view of the therapeutic animal crate, according to some embodiments.

FIG. 9 illustrates a diagrammatic view of the therapeutic animal crate in communication with a remote computing device, according to some embodiments.

FIGS. 10A and 10B illustrate a software application running on the remote computing device, according to some embodiments.

FIG. 11 illustrates an exploded profile view of the therapeutic animal crate, with an energized panel or energized mat located on the crate bottom, according to some embodiments.

FIG. 12 illustrates a front view of an energized panel, according to some embodiments.

### DETAILED DESCRIPTION

Although specific embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order-dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Component Index

100 – Crate
102 – Top wall
104 – Bottom wall
106 – Side wall
108 – Vent
110 – Electrical cord and plug
112 – Sliding panel
302 – Animal
402 – Locking mechanism
502 – Energized panel
502a – First energized panel
502b – Second energized panel
504 – Lights
506 – Energized mat
602 – Sensor
604 – Treat-dispensing device
606 – Camera
608 – Speaker
702 – Controller
704 – Fan
902 – Remote computing device
1002 – Software application

Current therapeutic technology for use on animals, while they are housed in a crate, depends on preprogrammed instructions to provide the therapy to the animal from components within the crate. Like humans, animals vary greatly in how they react to specific treatments. This variation can be seen in the type of animal, the breed of animal, the height, weight, or length of the animal, the color of the animal’s hair, or even the length of the animal’s hair, among other potential factors.

The available technology in the prior art does not take these variables into consideration. The disclosure herein is intended to teach an animal crate that is capable of taking into consideration the variations between different animals and tailoring the treatment provided to the animal receiving the treatment. For the purposes of this disclosure, crate, kennel, and housing are intended to refer to the same thing and are thus used interchangeably.

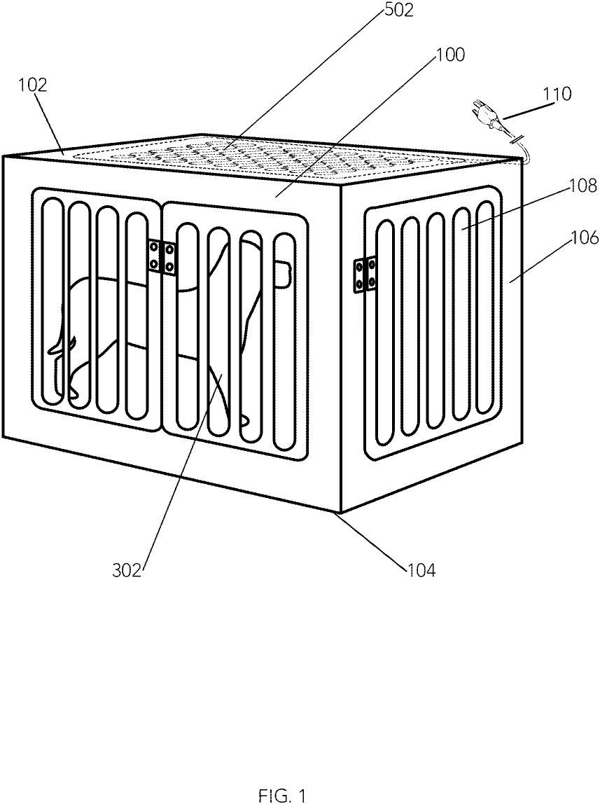

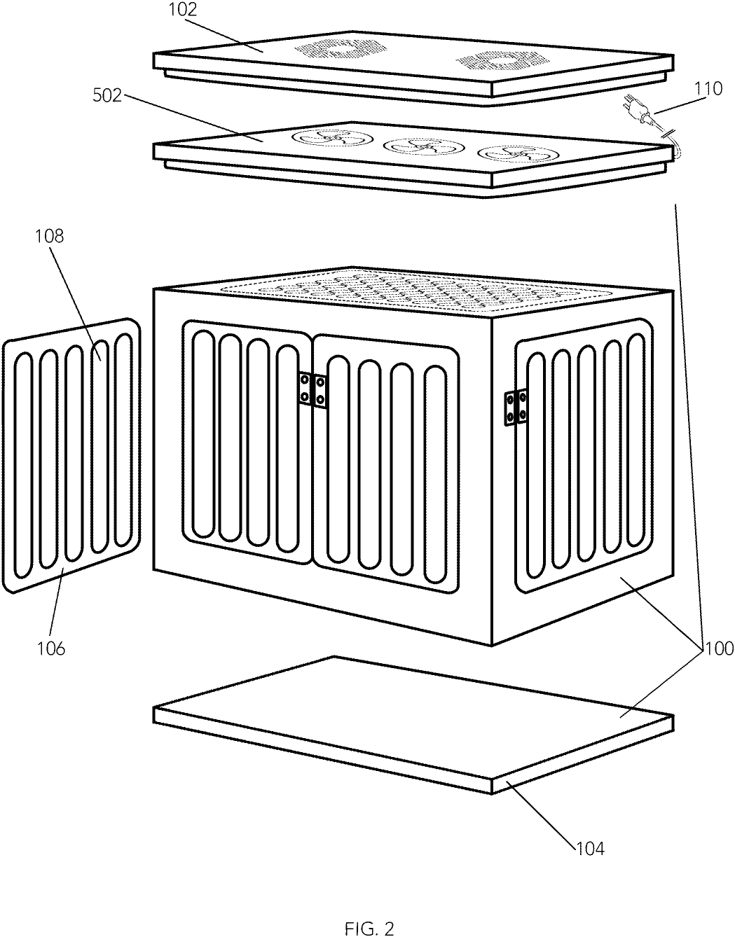

FIG. 1 illustrates a profile view of the therapeutic animal crate 100 with closed doors, and FIG. 2 illustrates an exploded profile view of the same. The crate 100 may consist of a top wall 102, a bottom wall 104, and at least one side wall 106 which connects the top wall 102 to the bottom wall 104, allowing the structure to stand freely. While shown as fixed rigidly to one another in FIG. 1, the top wall 102, bottom wall 104, and at least one side wall 106 may be coupled detachably to one another. This would allow the structure to fold down on itself, or collapse, to facilitate transportation of the device while not in use. The housing may be fixed in place, or it may be capable of transportation while in its constructed form. Additionally, while doors are shown in the side walls 106 of the crate 100, if it were so desired, the doors could be placed on the top wall 102 for lowering an animal 302 into the crate 100, or the bottom wall 104 for placing the crate 100 around an already positioned animal 302.

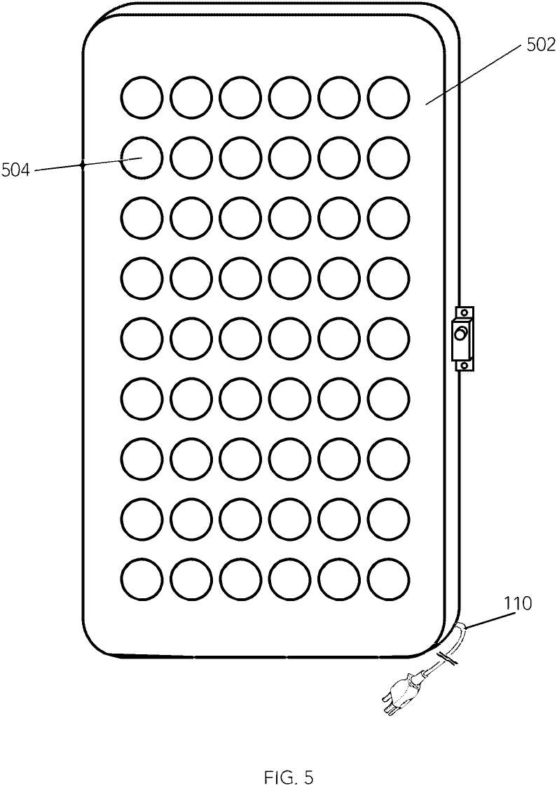

Any, all, or none of the top wall 102, bottom wall 104, and at least one side wall 106 may have vents 108. In some examples, the therapeutic animal crate 100 includes an energized panel 502 (see FIG. 5-7B). This panel may be high-powered, and heat is a natural by-product of high-power processes. These vents 108 allow air to flow freely, which helps to both cool down components within the therapeutic animal crate 100, such as the energized panel 502 itself, as well as the animal 302 that is being treated.

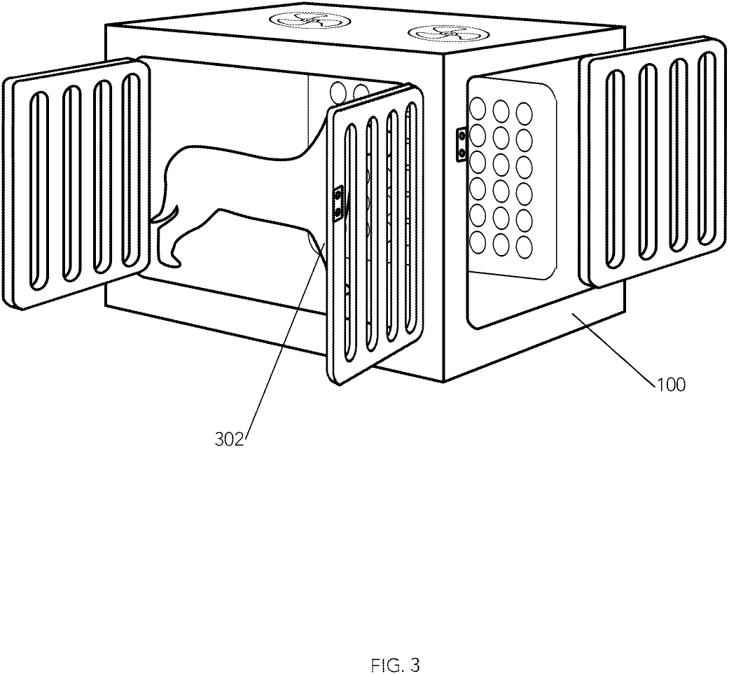

FIG. 3 illustrates a profile view of the therapeutic animal crate 100 with open doors. As is shown, an animal 302 may sit inside of the crate 100 while receiving treatment. The crate 100 may be sized such that animals 302 of any size are comfortable standing, sitting, or laying down within the interior. As such, the crate 100 may be constructed to any size that a user may need.

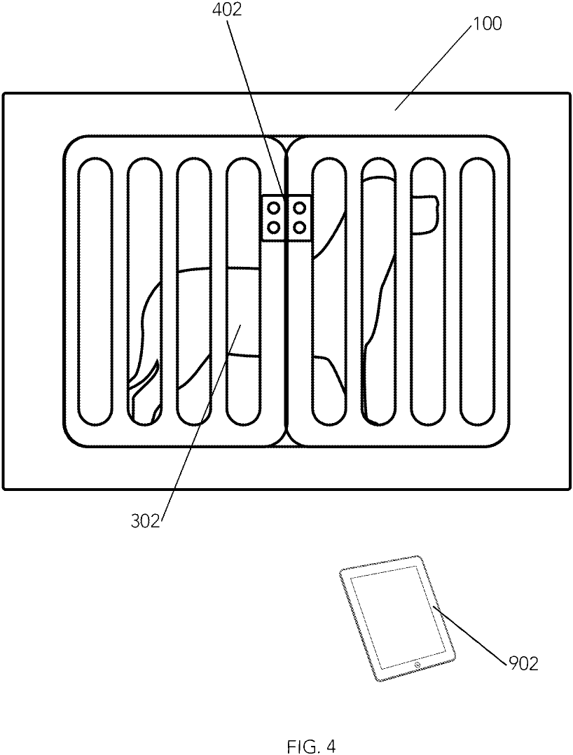

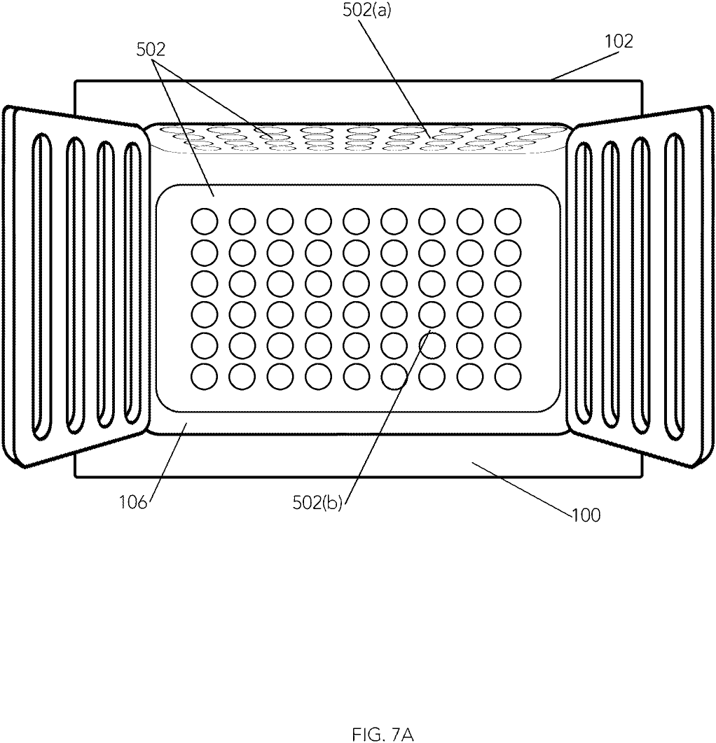

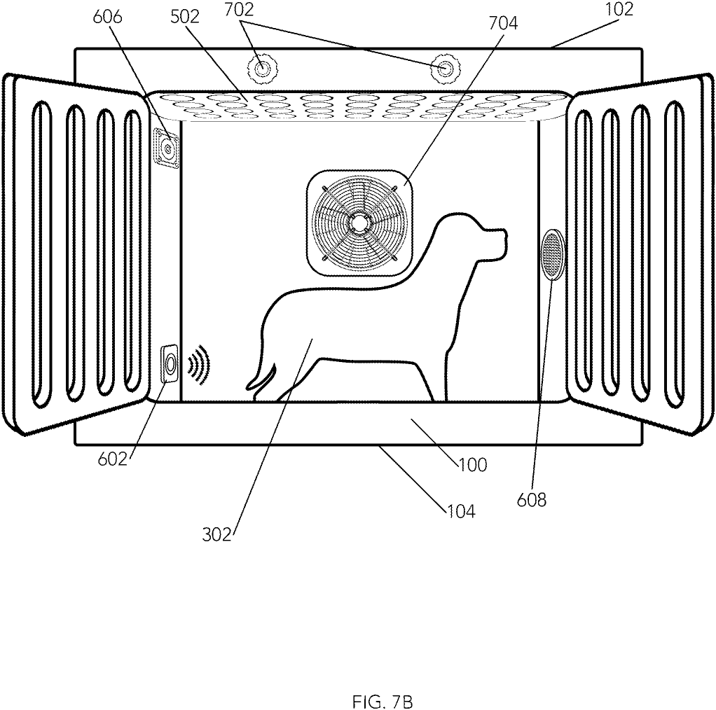

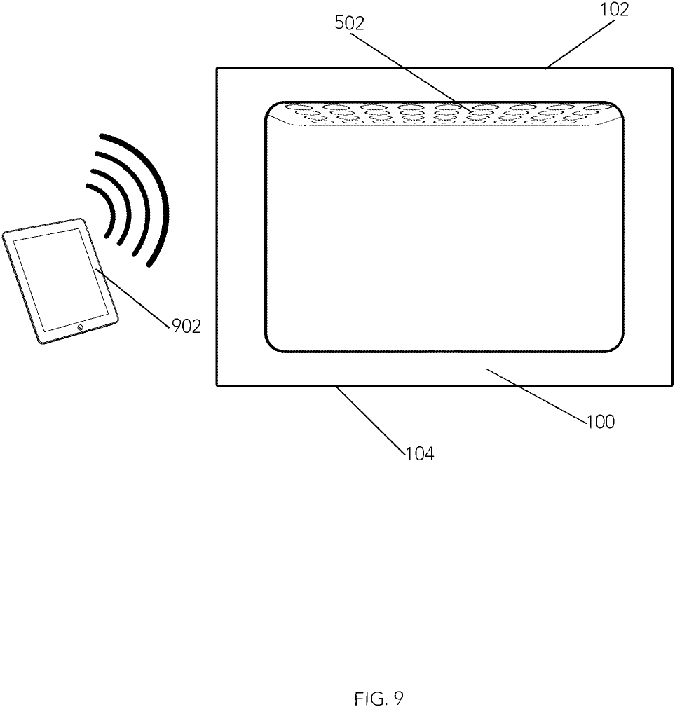

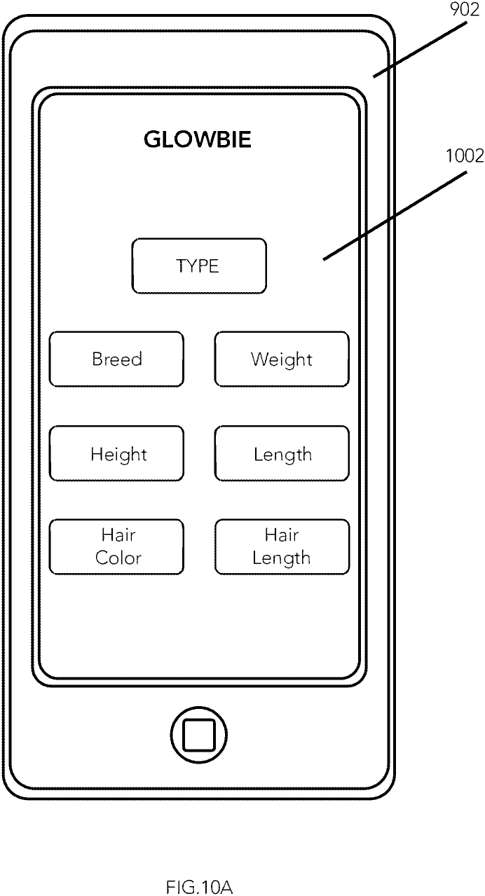

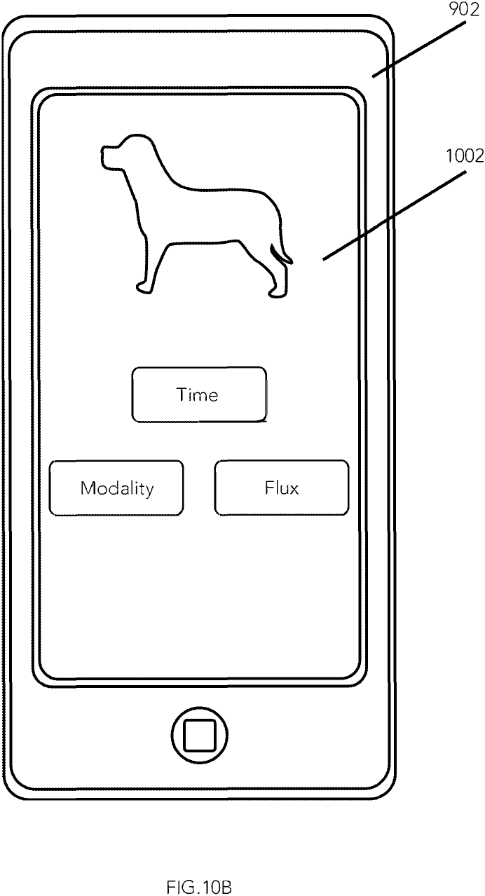

FIG. 4 illustrates a front view of the therapeutic animal crate 100 with closed doors. A locking mechanism 402 is seen on the doors of the crate 100. The locking mechanism 402 may be configured to prevent the animal 302 from exiting the crate 100, and in embodiments where the doors open inwards, the locking mechanism 402 may serve additionally to prevent the animal 302 from entering the crate 100 during unwanted times. This locking mechanism 402 may be controlled manually, from the controller 702 (as seen in FIG. 7B), or from a remote computing device 902 which will be discussed further in FIG. 9-10B.

FIG. 5 illustrates a front view of an energized panel 502. In some embodiments, the energized panel 502 is configured to provide light therapy to the animal 302. The energized panel 502 may deliver light in the visible red spectrum, as well as near-infrared (NIR) light in the invisible spectrum. According to some embodiments, the red light is provided at approximately 660 nanometers, and the NIR light is provided at approximately 850 nanometers, but values outside of these may also be used. According to some embodiments, ultraviolet (UV) light is also delivered by the energized panel 502. Additionally, lights within the visible spectrum other than red may be output by the energized panel 502. FIG. 5 shows an exemplary embodiment of lights 504 in a grid of round bulbs.

The energized panel 502 may be optimized for general veterinary applications. In other embodiments, the energized panel 502 can be adjusted by a user, either through a controller 702 (as discussed in FIG. 7B), or a remote computing device 902 (as discussed in FIG. 9-10B). According to some embodiments, the user is able to control the treatment time, the power (or irradiance) of the energized panel 502, the treatment modality, and the light flux (the measure of the perceived power of light). Not all of these features must be simultaneously controllable - in some embodiments, perhaps only the treatment time is controllable, and in others only the power of the energized panel 502.

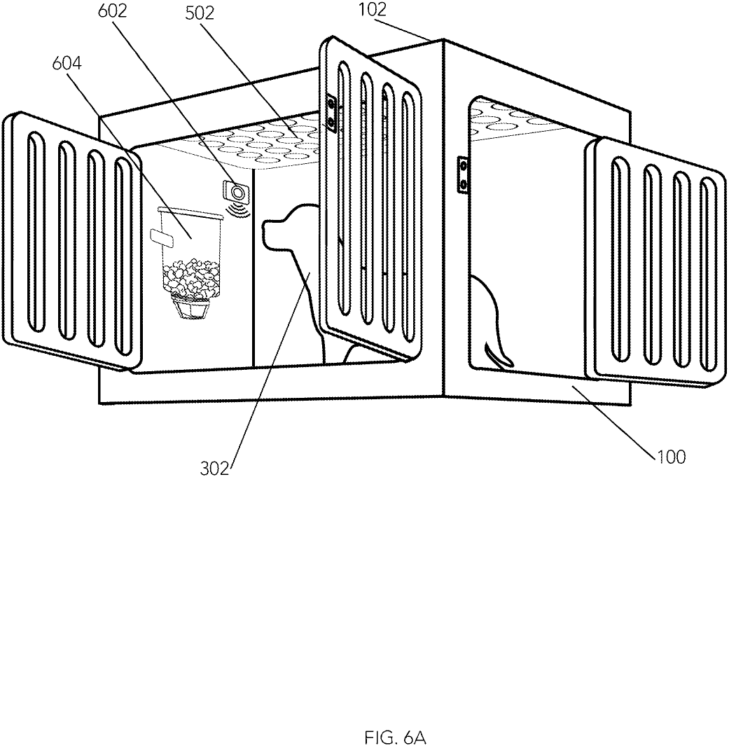

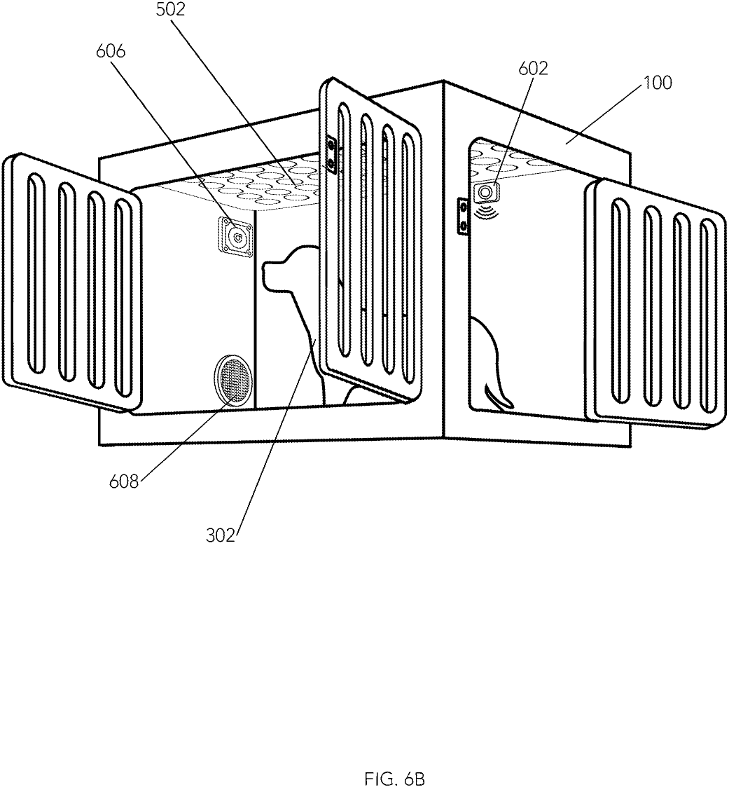

The therapy provided by the energized panel 502 may be described as photobiomodulation, phototherapy, photomedicine, or light therapy. The energized panel 502 may be able to deliver wavelengths of light that also sanitize the surfaces within the crate 100. According to some embodiments, the energized panel 502 may additionally provide pulsed electromagnetic field (PEMF) therapy and sound therapy. In other embodiments, these therapies are provided by other components within the crate 100 (as discussed in FIG. 6B). Each of these therapies may be used in conjunction or isolation.

FIGS. 6A and 6B illustrate profile views of the therapeutic animal crate 100 with open doors and various components that may be present within the system. With regards to FIG. 6A, a sensor 602 may be present. While the sensor 602 is located in the top corner of the crate 100, this positioning is exemplary only, and not the necessary position for the sensor 602. In some embodiments, the sensor 602 is used to measure a distance to the animal 302. This information may be used to control the intensity of power coming from the energized panel 502. As such, many embodiments will see the sensor 602 positioned near the location of the energized panel 502. However, there may be embodiments that use a combination of the sensor 602 and the energized panel 502, or multiple sensors 602, to triangulate the position of the animal 302 and use this information to control the power of the energized panel 502, while allowing the sensors 602 to be in positions that are not near the top wall 102 of the crate 100.

Also shown in FIG. 6A is a treat-dispensing device 604. This treat-dispensing device 604 may dispense a treat to the animal 302 based on an array of factors. In some embodiments, the treat may be dispensed on a fixed timeframe while the animal 302 is in the crate 100. In other embodiments, the treat may be dispensed at different times of treatment based on the total treatment time. In further embodiments, the treat-dispensing device 604 may receive input from the user based on the controller 702 (see FIG. 7B) or the remote computing device 902 (see FIG. 9-10B). In further embodiments still, the sensor 602 may be capable of detecting distress in the animal 302 based on noticed behavior, and dispense treats to help calm the animal 302 down.

With respect to FIG. 6B, a camera 606 may be positioned within the crate 100 to provide a view of the animal 302. As with the sensor 602 in FIG. 6A, the position shown is exemplary only, and the camera 606 may be placed anywhere within the crate 100. According to some embodiments, the camera 606 is configured to take pictures of the animal 302. These pictures may occur on a set timeframe or in response to a communication from the user via the controller 702 (FIG. 7B) or the remote computing device 902 (FIG. 9-10B). In other embodiments, the camera 606 is configured to take videos of the animal 302. The camera 606 may capture videos on a set timeframe, in response to a communication from the user, or throughout the entirety of treatment. The embodiments wherein the camera 606 takes still images and videos are not in isolation - the camera 606 may be configured to do both, or one or the other. In embodiments with a remote computing device 902 (FIG. 9-10B), the camera 606 may send still images or videos to said remote computing device 902.

The camera 606 may also be used as the sensor 602 component. In some embodiments, the camera 606 will measure the distance from the animal 302 and adjust the intensity or power of the energized panel 502 based on this distance. According to some embodiments, the camera 606 can also detect the type of animal 302, breed, height, length, hair color, or hair length, or any combination thereof, and adjust the treatment parameters based on this information.

Also shown in FIG. 6B is a speaker 608 in the lower corner of the crate 100. Once again, this positioning is exemplary only, and the speaker 608 may be placed anywhere within the crate 100. According to some embodiments, the speaker 608 is configured to provide sound therapy to the animal 302. This sound therapy may be structured into a preprogrammed therapy session, controlled by the user through either the controller 702 (FIG. 7B) or the remote computing device 902 (FIG. 9-10B), or started in response to noticeable distress of the animal 302, which may be found by the camera 606, the sensor 602, or a combination thereof.

The speaker 608 may also be configured to enable the user to communicate with the animal 302 from a remote location. According to some embodiments, the user is able to use a remote computing device 902 to speak to their animal 302, which may have additional calming effects. For example, if a user notices that their animal 302 is under distress from videos they receive on their remote computing device 902, and their embodiment does not have the functionality to respond to these distress signals, the user could use their remote computing device 902 to calm the animal 302 with their voice. This is in addition to any other potential reasons a user would have to communicate with the animal 302 being treated in the crate 100.

FIGS. 7A and 7B illustrate front views of the therapeutic animal crate 100 with the doors open. FIG. 7A shows two energized panels 502, a first energized panel 502a on the top wall 102, and a second energized panel 502b on one of the side walls 106. These positions are exemplary only, and the energized panels 502 may be located on any wall within the crate 100.

The first energized panel 502a and the second energized panel 502b have the full complement of the previously disclosed features, but these will be reiterated here. The energized panels 502 may be configured to provide light therapy, PEMF therapy, sound therapy, or any combination thereof. The two energized panels 502 do not need to have all of the capabilities as the other in the pair. The energized panels 502 may deliver light in the visible red spectrum, as well as NIR light in the invisible spectrum. According to some embodiments, the red light is provided at approximately 660 nanometers, and the NIR light is provided at approximately 850 nanometers, but values outside of these may also be used. According to some embodiments, ultraviolet (UV) light is also delivered by the energized panels 502. Additionally, lights within the visible spectrum other than red may be output by the energized panels 502.

The energized panels 502 may be optimized for general veterinary applications. In other embodiments, the energized panels 502 can be adjusted by a user, either through a controller 702 (as discussed in FIG. 7B), or a remote computing device 902 (as discussed in FIG. 9-10B). According to some embodiments, the crate 100 includes a user interface that enabled the user to control the treatment time, the irradiance of the energized panels 502, the treatment modality, the light flux, the PEMF therapy settings, and the sound therapy settings. Not all of these features must be simultaneously controllable - in some embodiments, perhaps only the treatment time is controllable, and in others only the power of the energized panels 502.

The therapy provided by the energized panels 502 may be described as photobiomodulation, phototherapy, photomedicine, or light therapy. The energized panels 502 may be able to deliver wavelengths of light that also sanitize the surfaces within the crate 100.

While two energized panels 502 are shown in FIG. 7A, it is understood this shows that any number of energized panels 502 as can fit along the walls of the crate 100 may be incorporated into the system.

With respect to FIG. 7B, a fan 704 is shown on one of the side walls 106 of the crate 100. This position is exemplary only, and the fan 704 may be on any wall of the crate 100, including a wall that already has the energized panel 502, in exemplary embodiments with the energized panel 502 and the fan 704 of sizes defined for both to be installed on the same wall. The fan 704 may be configured to dissipate heat that is building up within the crate 100 due to the heat radiating from the energized panel(s) 502. According to some embodiments, the fan 704 is configured to cool down the animal 302 inside of the crate 100.

Also shown in FIG. 7B is a potential embodiment of a controller 702 coupled to the crate 100. While FIG. 7B shows two such controllers 702, the crate can be configured with any number of controllers 702 as desired, from no controllers to as many controllers 702 as can fit on the crate 100. According to some embodiments, the controller 702 is configured to allow the user to control the energized panel(s) 502 or any components (i.e., the sensor 602, the camera 606, the speaker 608, the fan 704, etc.) within the crate 100. The controller(s) 702 may control the treatment time, treatment modality, light flux, PEMF therapy settings, sound therapy settings, or combinations thereof, of the energized panel(s) 502. The controller(s) 702 may also control such aspects as turning off and on the fan 704, turning off and on the camera 606, turning off and on the speaker 608, or turning off and on the sensor 602.

While the individual components shown in FIGS. 6A-7B, as well as those disclosed in the preceding paragraph are shown in separate embodiments, it should be understood that any combination of these components, including all components (i.e., the speaker 608, the sensor 602, the camera 606, the controller 702, the heater or heating element, the mat, and the treat dispensing device) or none of the components, may be present inside of the crate 100.

Additionally, any or all of the components (i.e., the speaker 608, the sensor 602, the camera 606, and the heater or heating element) may be integrated into the energized panel. These components may be controlled by a user either through the use of the controller 702 or the software application 1002 running on a remote computing device 902 as seen in FIG. 9-10B.

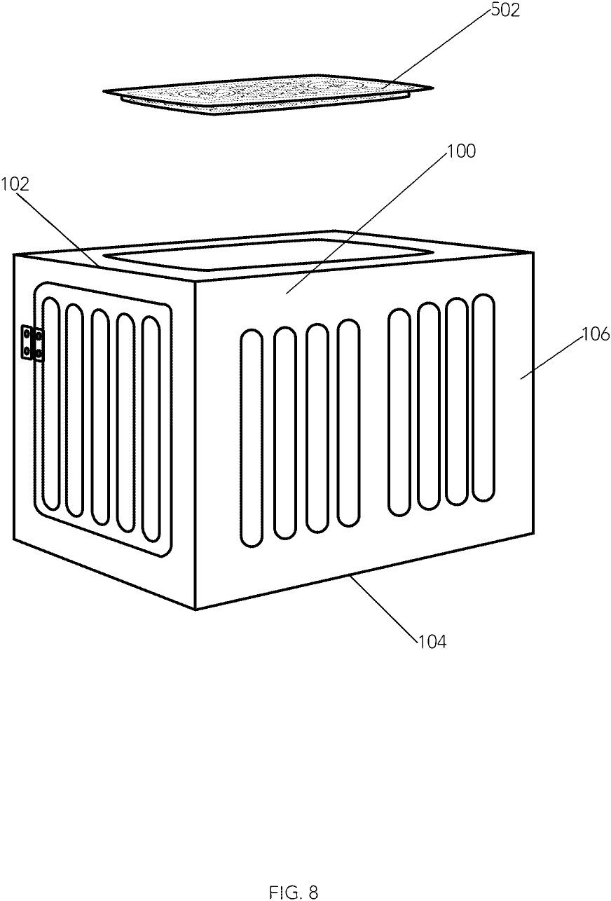

FIG. 8 illustrates a profile view of the therapeutic animal crate 100 with closed doors. According to some embodiments, the energized panel(s) 502 are detachable from the crate 100. While FIG. 8 shows a door integrated into a side wall 106, in some embodiments there is no door on the side wall 106, and the entrance to the interior is provided by detaching the energized panel 502 from the wall on which it attaches. Additionally, while only one energized panel 502 is shown detaching from the crate 100, it should be understood that any additional energized panels present in the device may also be detachable. Finally, while the energized panel 502 is shown detaching from the top wall 102 of the crate 100, it should also be understood that the energized panel 502 may be detachably coupled to any of the walls of the crate 100.

FIG. 9 illustrates a diagrammatic view of the therapeutic animal crate 100 in communication with a remote computing device 902. FIGS. 10A and 10B illustrate two exemplary embodiments of a software application 1002 running on said remote computing device 902.

The remote computing device 902 may be any device that enables communication over a span of distance, i.e., communication that does not necessitate proximity to the therapeutic animal crate 100, such as a phone or tablet device.

In some embodiments, the software application 1002 allows for user input. According to some embodiments, this user input may include the type of animal 302 entering the crate 100, the breed of the animal 302, the weight of the animal 302, the height of the animal 302, the length of the animal 302, the hair color of the animal 302, the hair length of the animal 302, or combinations thereof, as shown in FIG. 10A. These factors can then be used to calculate an optimal therapeutic treatment, and then communicate the instructions of said therapeutic treatment to the therapeutic animal crate 100 remotely. These inputs may be used in tandem with the information garnered by a sensor 602 or camera 606 as disclosed in FIGS. 6A and 6B.

According to some embodiments, the software application 1002 is configured to do any or all of the things the controller 702 has been described to do, such as controlling the treatment time, treatment modality, light flux, PEMF therapy settings, sound therapy settings, or combinations thereof of the energized panel(s) 502. An exemplary image of what a user interface generated by the software application 1002 might look like when controlling these features is shown in FIG. 10B. Additionally, the software application 1002 may allow control of optical intensity delivered by the energized panel, the light wavelengths delivered by the energized panel, and the type of therapy delivered by the energized panel, and combinations thereof. The software application 1002 may also enable to user to control the sensor 602, the camera 606, the speaker 608, the fan 704, the heater or heating element, or the mat, or any combination thereof if said components are present in the therapeutic animal crate 100. In some embodiments. The software application 1002 controls functional elements such as turning off and on the fan 704, turning off and on the camera 606, turning off and on the speaker 608, or turning off and on the sensor 602. Finally, the software application 1002 may be programmed to control the amount of time on a timer present in the crate 100 or to lock or unlock the locking mechanism 402 remotely on any of the present doors of the crate 100.

Additionally, the software application 1002 may be capable of tracking treatments over time. In some embodiments, this information is shared automatically, or shared via input from the user, with a veterinarian. According to some embodiments, the software application 1002 enables communication with the veterinarian. The software application 1002 may also include a community feature, where users of the therapeutic animal crate 100 can communicate with one another, or ask questions.

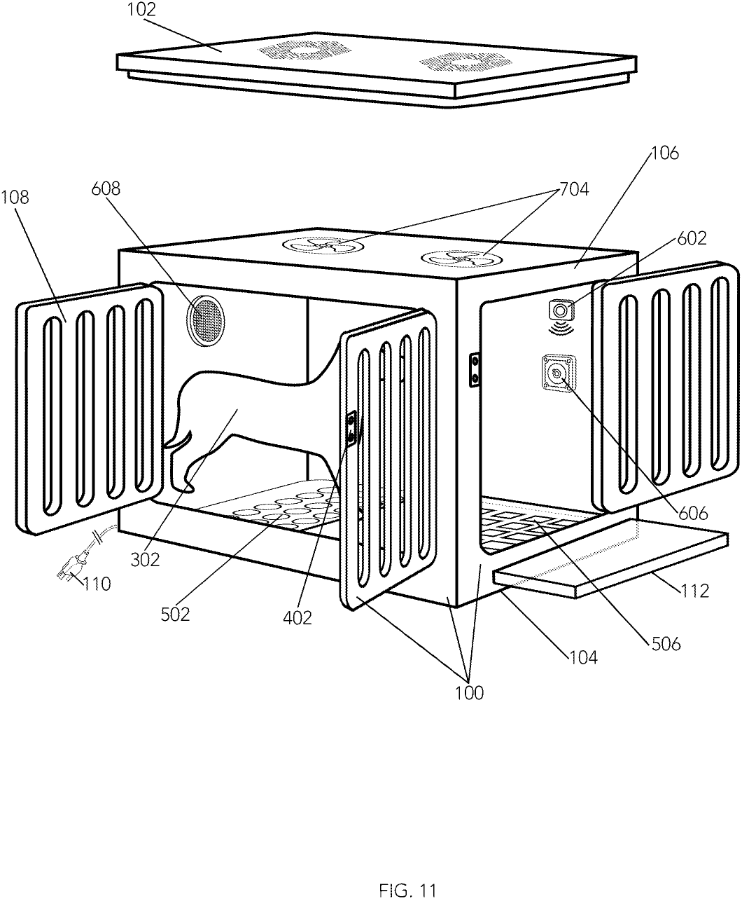

FIG. 11 illustrates an exploded profile view of the therapeutic animal crate, with an energized panel located on the crate bottom, according to the example embodiment shown. The structure of the animal crate 100 is comprised of top wall 102, bottom wall 104, side walls 106, and door latch 402 to secure the animal 302. In this embodiment, the energized panel 502 is placed underneath the animal 302 and the two are separated by sliding panel 112, commonly constructed from transparent acrylic plastic. Although the figure depicts the bottom wall 104 coupled to a combination energy panel 502 with round lights 504 or an energized mat 506 with square output elements, this is combination panel represents the ability to select either an energy panel 502 or an energized mat 506 for coupling to the bottom wall 104. Fans 704 are included for air movement and animal 302 cooling. Power is provided through cord 110 and the therapy and crate 100 environments can be controlled through information and feedback of the sensor 602, camera 606, and speaker 608. The crate 100 may also include an energized mat 506 installed on top of the bottom wall 104. According to some embodiments, the energized mat 506 is a PEMF mat that is configured to deliver PEMF therapy to the animal 302. In some embodiments, the energized mat 506 is an integrated mat that emits therapeutic light of varying optical intensity. According to other embodiments, the energized mat is configured with temperature controls and an electrical resistance heater to provide heat to the animal 302. Other embodiments are an energized mat configured with a temperature controller and means known in the art of heating and cooling technology to provide evaporative cooling capabilities (e.g., cooling provided by wall temperature reduction owing to the phase conversion of the heat-transfer fluid, continuous circulation of heat transfer fluid through a cooling heat exchanger, and/or sealed, pressurized expansion and compression of refridgerant). The distinct capabilities of the energized mat 506 are depicted in FIG. 11 as square elements in contrast to the round elements representing lights 504.

The energized mat 506 is separated by sliding panel 112, commonly constructed from transparent acrylic plastic. Additionally, while also not illustrated in any of the figures, the crate 100 may include a heater or other heating element installed along any of the walls. The heater or heating element may be configured to deliver heat to the animal 302, both to attract the animal 302 to the interior of the crate 100, as well as the heat the animal 302 and the interior of the crate 100, should the energized panel(s) 502 not be providing enough heat.

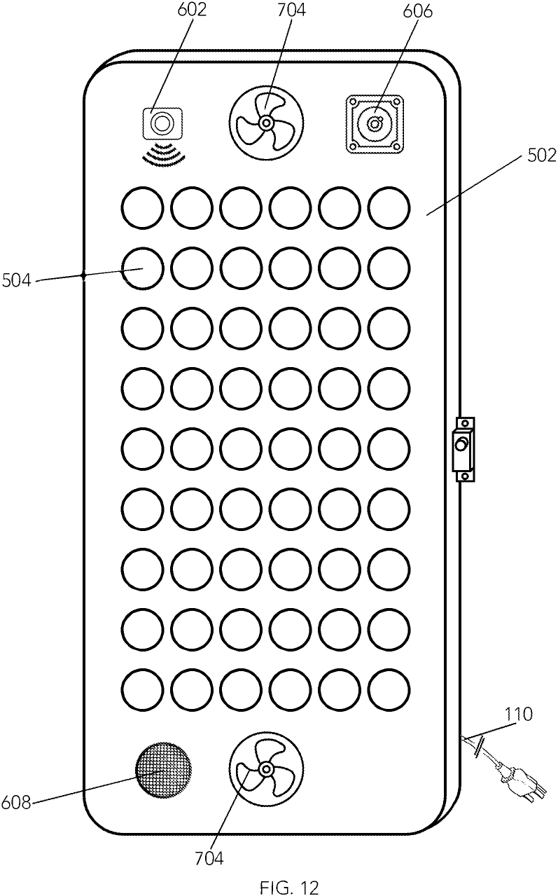

To optimize investments in animal confinement and preserve animal comfort with a continuous housing situation, the crate or kennel can be built with an energized panel that incorporates the electronic peripherals required for therapy optimization. FIG. 12 illustrates a front view of an energized panel with such exemplary incorporations. According to some embodiments, the energized panel 502, powered through electric cord 110 can contain the therapy lights 504, fans 704, a single or multiples of sensor 602, a camera 606, and a speaker 608. The energized panels 502 may be configured to provide light therapy, PEMF therapy, sound therapy, or any combination thereof.

### INTERPRETATION

None of the steps described herein are essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section.

To increase the clarity of various features, other features are not labeled in each figure.

The various features and processes described above may be used independently of one another or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, parallel, or some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless expressly stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless expressly stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only and are not intended to limit the scope of the inventions disclosed herein. Thus, nothing in the foregoing description implies that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. A therapeutic crate system providing therapy to an animal, comprising:
a therapeutic crate including a top wall, a bottom wall opposite the top wall, and side walls extending between the top wall and the bottom wall;
at least a first energized panel coupled to at least one wall selected from the group consisting of the top wall, the bottom wall, at least one of the side walls, and combinations thereof, the first energized panel configured to deliver a type of therapy selected from the group consisting of phototherapy, pulsed electromagnetic field (PEMF) therapy, and sound therapy;
a controller operatively coupled to the first energized panel; and
a non-transitory computer-readable media, executable by a processor selected from the group consisting of a remote computing device, the first energized panel, the controller, and combinations thereof, the non-transitory computer-readable media configured to cause the processor to carry out the step of obtaining information related to an animal characteristic selected from the group comprising a hair length, a hair color, and combinations thereof,
wherein the information is provided by a component selected from the group consisting of the remote computing device, the controller, a camera, and combinations thereof, and
wherein the camera is coupled to at least one wall selected from the group consisting of the top wall, the bottom wall, at least one of the side walls, and combinations thereof.

2. The therapeutic crate system of claim 1, wherein:
the first energized panel includes programmable elements, and
the non-transitory computer-readable media is further configured to cause the processor to carry out the step of adjusting a programmable element of the first energized panel selected from the group consisting of treatment time, treatment modality, light flux, PEMF therapy settings, sound therapy settings, and combinations thereof in response to obtaining information related to the animal characteristic.

3. The therapeutic crate system of claim 1, wherein:
the first energized panel includes programmable elements, and
the non-transitory computer-readable media is further configured to cause the processor to carry out the step of adjusting a programmable element of the first energized panel selected from the group consisting of optical intensity, light wavelengths, a type of therapy, and combinations thereof in response to obtaining information related to the animal characteristic.

4. The therapeutic crate system of claim 1, further comprising a sensor coupled to at least one wall selected from the group consisting of the top wall, the bottom wall, at least one of the side walls, and combinations thereof, the sensor configured to determine a distance between the animal and the first energized panel.

5. The therapeutic crate system of claim 4, wherein the first energized panel is configured to adjust light intensity based on the distance between the animal and the sensor.

6. The therapeutic crate system of claim 1, wherein the first energized panel is configured to detach from the wall.

7. The therapeutic crate system of claim 1, further comprising an energized mat coupled to the bottom wall.

8. The therapeutic crate system of claim 1, further comprising a second energized panel located along at least one wall selected from the group consisting of the top wall, the bottom wall, at least one side wall, and combinations thereof, wherein the first energized panel and the second energized panel are not located on a same wall.

9. The therapeutic crate system of claim 8, wherein the second energized panel delivers a therapy selected from the group consisting of PEMF therapy, sound therapy, and combinations thereof.

10. The therapeutic crate system of claim 1, wherein the non-transitory computer-readable media is further configured to cause the processor to perform the step of changing an amount of time on a timer.

11. The therapeutic crate system of claim 1, wherein the non-transitory computer-readable media is further configured to cause the processor to perform the step of remotely unlocking a locking mechanism.

12. The therapeutic crate system of claim 1, further comprising a speaker coupled to the therapeutic crate, wherein:
the speaker is configured to emit a sound, and
the non-transitory computer-readable media is further configured to cause the processor to perform the step of emitting, via the speaker, a sound selected from the group consisting of communication to an animal located inside the therapeutic crate, sound therapy, and combinations thereof.

13. The therapeutic crate system of claim 1, wherein the therapeutic crate is configured to collapse.

14. The therapeutic crate system of claim 1, wherein the non-transitory computer-readable media is further configured to cause the processor to carry out the step of obtaining information related to a breed of the animal.

15. A method of treating an animal, comprising:
obtaining a therapeutic crate including a top wall, a bottom wall opposite the top wall, side walls extending between the top wall and the bottom wall, and an energized panel, the energized panel configured to deliver a type of therapy selected from the group consisting of phototherapy, pulsed electromagnetic field (PEMF) therapy, and sound therapy;
disposing the animal in the therapeutic crate; and
obtaining information related to an animal characteristic selected from the group comprising a hair length, a hair color, and combinations thereof,
wherein the information is provided by a component selected from the group consisting of a remote computing device, a controller, a camera, and combinations thereof, and
wherein the camera is coupled to at least one wall selected from the group consisting of the top wall, the bottom wall, at least one of the side walls, and combinations thereof.

16. The method of claim 15, further comprising adjusting a programmable element of the energized panel selected from the group consisting of treatment time, treatment modality, light flux, PEMF therapy settings, sound therapy settings, and combinations thereof in response to obtaining information related to the animal characteristic.

17. The method of claim 15, further comprising adjusting a programmable element of the energized panel selected from the group consisting of optical intensity delivered by the energized panel, light wavelengths delivered by the energized panel, a type of therapy delivered by the energized panel, and combinations thereof in response to obtaining information related to the animal characteristic.

18. The method of claim 15, further comprising changing an amount of time on a timer.

19. The method of claim 15, further comprising unlocking, remotely, a locking mechanism.

20. The method of claim 15, wherein the crate further includes a speaker coupled to at least one wall selected from the group consisting of the top wall, the bottom wall, at least one side wall, and combinations thereof, the method further comprising emitting, via the speaker, a sound selected from the group consisting of communication to an animal located inside the crate, sound therapy, and combinations thereof.
